(19) United States
(12) Patent Application Publication
(10) Pub. No.: US 2018/0020707 A1
(43) Pub. Date: Jan. 25, 2018

(54) A NATURAL SWEETENING COMPOSITION OF LUO HAN GUO AND APPLE

(71) Applicant: MONTERO GIDA SANAYI VE TICARET A.S., Istanbul (TR)

(72) Inventors: Ahmet TOKSÖZ, Istanbul (TR); Zafer TOKSÖZ, Istanbul (TR); Onur MUTLU, Istanbul (TR); Ali TÜRKYILMAZ, Istanbul (TR); Gülay YELKEN, Istanbul (TR); Merve ZAN, Istanbul (TR)

(21) Appl. No.: 15/546,471

(22) PCT Filed: Jan. 26, 2016
(86) PCT No.: PCT/EP2016/051493

§ 371 Date: Jul. 26, 2017

(30) Foreign Application Priority Data
Jan. 27, 2015 (TR) 2015/00903
Mar. 6, 2015 (TR) 2015/02736

(51) Int. Cl. A23L 27/12; A61K 47/46; A61K 9/00; A23L 2/60; A23K 20/10

(52) U.S. Cl. CPC A23L 27/12; A23L 2/60; A23K 20/10; A61K 9/0053; A61K 47/46; A23V 2002/00

(57) ABSTRACT

The present invention relates to a natural sweetening composition comprising apple concentrate having a median particle size (d50) greater than 100 μm and Luo Han Guo concentrate having a median particle size (d50) greater than 1 μm and use in beverage, food, pharmaceutical, oral, dietetic, veterinary or tobacco products thereof.

### FIELD OF INVENTION

The present invention relates to a natural sweetening composition comprising apple concentrate having a median particle size (d50) greater than 100 μm and Luo Han Guo concentrate having a median particle size (d50) greater than 1 μm and use in beverage, food, pharmaceutical, oral, dietetic, veterinary or tobacco products thereof.

### BACKGROUND OF INVENTION

Luo Han Guo (luohanguo) refers to the fruit of Siraitia grosvenori, formerly called Momordica grosvenori, a member of the Curcubitaceae. The plant is cultivated for its fruit Luo Han Guo, whose extract is nearly 300 times sweeter than sugar and has been used in China as a natural low-calorie sweetener and in traditional Chinese medicine to treat diabetes and obesity. Luo Han Guo is popularly considered a longevity aid and is used to balance heat buildup caused by internal conditions, life-forces, or external heat. It is used as an expectorant and antitussive to treat lung congestion, cough, other respiratory ailments, and sore throat. It also is used for constipation and chronic enteritis. Luo Han Guo is a low-caloric, low-glycemic food used as a natural sweetener in beverages and food.

Luo Han Guo is collected as a round green fruit that turns brown upon drying. The sweet taste of Luo Han Guo comes primarily from mogrosides, a group of terpene glycosides, present at the level of about 1% of the fleshy part of the fruit. Both the fresh and dried fruits are extracted to yield a powder that is 80% or more mogrosides. The mogrosides have been numbered, 1-5, and the main component is called mogroside-5, previously known as esgoside. Other, similar compounds from Luo Han Guo have been labeled siamenoside and neomogroside. The mixed mogrosides are estimated to be about 300 times as sweet as sugar by weight, so that the 80% extracts are nearly 250 times sweeter than sugar; pure mogrosides 4 and 5 may be 400 times as sweet as sugar by weight.

A process for making a useful sweetener from Luo Han Guo was patented in 1995 by Procter and Gamble Company in U.S. Pat. No. 5,411,755. As described in the patent application, the fruit itself, though sweet, has too many additional flavors that would make it unsuitable for widespread use as a sweetener, so P&G developed a method for processing it to eliminate the undesired flavors. In the P&G process, the fresh fruit is picked before ripening and allowed to complete its ripening during storage so that processing begins with the just-ripe fruit. The peel and seeds are then removed, and the mashed fruit becomes the basis of a concentrated fruit juice or puree that can be used in food manufacturing. Further processing involves using solvents to remove volatile and off-flavor components. Numerous sugar substitutes derived from Luo Han Guo by similar processes that isolate the sweet compounds are now readily available for manufacturing and for kitchen use.

Recent work on Luo Han Guo includes investigation of the antioxidant activity of the mogrosides (Shi H, et al., Antioxidant property of fructus momordicae extract, 1996 Biochemistry and Molecular Biology International 1996; 40 (6): 1111-1121.) and their potential use as cancer prevention compounds (Konoshima T and Takasaki M, Cancer-chemopreventive effects of natural sweeteners and related compounds, Pure Applied Chemistry 2002; 74(7): 1309-1316.). This suggested effect is based on the understanding that antioxidants can produce significant reversal or suppression of the early stage of cancer development, which has been an area of particular interest for tea drinking (Katiyar SK and Mukhtar H, Tea antioxidants in cancer chemoprevention, Journal of Cellular Biochemistry, Supplement 1997; 27: 59-67.). Further, Luo Han Guo and its sweetening component are often mentioned in relation to diabetes and obesity, because it can substitute for caloric sugars normally consumed in the diet.

Apple concentrate is a flavouring, colouring and sweetening extract that contains the fruits own natural sugars. It also contains the fruit acids, minerals and secondary plant substances of apples—the polyphenolics which are nowadays regarded as positive health promoters. These polyphenolic substances are attributed with anti-oxidative and cancer-protective properties and are also regarded to have a positive effect on blood glucose and hypertension as well as lowering the cholesterol level. Further it is presumed that the positive nutritional and physiological effects of apple minerals and vitamins are associated with these secondary plant substances. Apple concentrate is a natural alternative to one of our most popular but problematic food commodities, the sucrose. For both food producers and consumers they offer various advantages to traditional sugar. Natural apple concentrate made from de-juiced and carefully dried apples that reflect the characteristic sugar spectrum, balancing the nutritional and physiological profiles with the consumers association of apple as symbol for health and vitality. The apple concentrate does not contain sucrose but a very high content of fructose.

Due to these neutral properties the potential application for natural sweetener composition is diverse, so this high quality sweetener can be used to replace sucrose in virtually every single food. Although sucrose, provides the most desirable taste to consumers, it is caloric and unhealthy. Furthermore, the natural sweetener composition is process friendly with virtually the same sweetening power as traditional sugar. But to be able to claim that you are using “natural sweetener” instead of sucrose provides a consumer friendly image and a positive sales argument. The carbohydrate fructose found in the natural sweetener composition is generally recognised as diabetic sweeteners, as compared to sucrose they are sugar substitutes and metabolised independent of insulin.

It has been also known numerous natural sweeteners are non-caloric; however, they exhibit sweet tastes that have different temporal profiles, maximal responses, flavor profiles, mouthfeels, and/or adaptation behaviors than that of sugar. Because of these differences, in a food or beverage, causes an unbalanced temporal profile and/or flavor profile. In addition to the difference in temporal profile, natural sweeteners generally exhibit:

(i) lower maximal response than sugar,

(ii) tastes including bitter, metallic, cooling, astringent, licorice-like taste, etc.,

(iii) sweetness which diminishes on iterative tasting.

It is well known to those skilled in the art of food/beverage/pharmaceutical composition that the sweetener in a composition requires balancing of the flavor and other taste components. If the taste profile of natural sweeteners could be modified to impart specific desired taste characteristics to be more sugar-like, the type and variety of compositions that may be prepared with that sweetener would be expanded significantly. Accordingly, it would be desirable to selectively modify the taste characteristics of natural sweeteners.

Another important point for the food/beverage/pharmaceutical composition is processability. The natural sweetening composition must be easily handled, mixed with excipients even having a larger particle size and must be processed into beverage, food, pharmaceutical, oral, dietetic, veterinary or tobacco products without the known and expected problems such as segregation or de-mix during the process steps. It is usually related with the particle size and particle size distribution. For the natural sweetener compositions, it is not always easy to achieve content uniformity, aggregation and large particle size can be occurred during the process.

In this invention it is surprisingly found that, specific particle size of each apple and Luo Han Guo concentrates also gives homogeneity and high flowability properties to the composition during the process. And thus change the taste balance of a food or beverage or pharmaceutical compositions. They are slower in onset and longer in duration than the sweet taste produced by sucrose.

### DESCRIPTION OF THE INVENTION

Health trends have promoted an increased use of natural sweeteners in consumer diets. Amongst the many possibilities available to product developers is the option to enrich foods with “healthy ingredients” or perhaps to exchange one or more ingredients of the recipe in order to optimise the nutritional profile.

For instance foods, which do not include additives or sugar, are perceived by the consumer, as natural and healthy. Despite their immense popularity, sweeteners and particularly sucrose recently gained bad publicity because of health concerns. That's why fruit sweeteners are becoming more and more important as a natural alternative to sucrose as they contain the fruit's own sugar spectrum.

Of increasing nutritional and physiological importance to today's consumer is the glycemic index (GI) that is much lower for fructose compared to that of glucose. Using glucose as the standard base, with a GI of 100, fructose has only a GI of around 20. The GI measures the glycemic response (an indication of the rate at which the blood glucose level rises and how it is sustained over time) after ingestion of carbohydrate foods. The consumption of food with low GI, such as fruit and vegetables, results in a slow increase of the blood glucose level. Food with a high GI, such as sucrose and white bread, results in a quick response of blood glucose insulin levels and therefore should not only be avoided by diabetics, but also by the growing number of health conscious consumers as there is growing evidence that over long time, a diet based on high-carbohydrate low-glycemic foods is beneficial towards health by preventing chronic diseases such as diabetes, coronary heart disease and possibly cancer.

In present invention, the phrase “concentrate” includes any apple or Luo Han Guo product that is solid comprising “extract”, “concentrated extract”, “dried fruit”, “de-juiced fruit”, “lyophilized”, “spray dried”, “crystallized” or mixtures thereof.

In present invention, the concentrates comprising fructose, aromas, flavorings, vitamins, minerals, nutrients, concentrated juices, pieces of pulp and other remnants or mixtures thereof.

The main object of the present invention is to obtain adequate content uniformity and desired particle size distribution of natural sweetening composition comprising apple concentrate and Luo Han Guo concentrate and improved processes. In this invention, the natural sweetening composition has homogeneity and high flowability properties during the process.

Another main object of this invention is to obtain a sweetener composition that has sweeter taste because of the synergy between apple concentrate and Luo Han Guo concentrate. This invention relates to a natural sweetening composition comprising apple concentrate and Luo Han Guo concentrate, the composition is exhibiting synergy, i. e. providing greater sweetness than would be expected from simple summation of the sweetness contributed by the component sweetening agents.

Another main object of the present invention is to obtain a sweetener composition that does not increase the glycemic index. This invention provides a sweetener composition, which does not raise the glycemic index and provides the advantage of production simplicity.

Another main object of the present invention is to obtain a sweetener composition that is natural and healthy. Also it also would be desirable to improve the taste of ingestible compositions that include natural ingredients to promote their use and the resulting health benefits. In this invention, the natural sweeteners apple concentrate and Luo Han Guo concentrate do not carry any glycemic index potential. Thus, a product is obtained which can be used by those who are in diets or by the diabetics.

The present invention provides a novel natural sweetening composition comprising apple concentrate having a median particle size (d50) greater than 100 μm and Luo Han Guo concentrate having a median particle size (d50) greater than 1 μm which overcomes the above described problems in prior art and have additive advantages over them.

In an embodiment, the natural sweetening composition comprising; apple concentrate having a median particle size (d50) is between 100 and 500 μm and Luo Han Guo concentrate having a median particle size (d50) is between 1 and 50 μm.

In an embodiment, the natural sweetening composition of this invention comprises apple concentrate having a median particle size (d50) greater than 100 μm, preferably greater than 250 μm, more preferably greater than 300 μm and having particle size of d(90) greater than 600 μm, preferably greater than 750 μm.

In a more preferably embodiment, the natural sweetening composition of this invention comprises apple concentrate having a median particle size (d50) greater than 300 μm and having particle size of (d90) greater than 750 μm.

In another embodiment, the natural sweetening composition of this invention comprises Luo Han Guo concentrate having a median particle size (d50) greater than 1 μm, preferably greater than 5 μm, more preferably greater than 10 μm and having particle size of (d90) greater than 20 μm, preferably greater than 30 μm.

In a more preferably embodiment, the natural sweetening composition of this invention comprises Luo Han Guo concentrate having a median particle size (d50) greater than 10 μm and having particle size of (d90) greater than 30 μm.

As used here in, “particle size distribution” is defined by the cumulative volume size distrubition as tested by a conventionally accepted method which is the laser diffraction method determined by the equipment of Malvern Mastersizer 2000. “Median particle size” is defined by “d50”, the diameter where fifty percent of the distribution has a smaller particle size and “d90” means that the the diameter where ninety percent of the distribution has a smaller particle size. “Desired particle size distribution” is defined by the ratio between the median particle size (d50) and the particle size at (d90).

In order to achieve the unexpected good results of content uniformity the natural sweetening composition of this invention the apple concentrate and Luo Han Guo concentrate must have a desired particle size distribution.

The natural sweetening composition of the present invention comprising; apple concentrate having a particle size ratio of (d50) to (d90) which is between 0.30 and 0.80; Luo Han Guo concentrate having a particle size ratio of (d50) to (d90) which is between 0.20 and 0.70.

According to this embodiment, this ratio of (d50) to (d90) of apple concentrate must be between 0.30 and 0.80, preferably between 0.35 and 0.70, more preferably between 0.40 and 0.60, and most preferably between 0.40 and 0.50.

According to this embodiment, this ratio of (d50) to (d90) of Luo Han Guo concentrate must be between 0.20 and 0.70, preferably between 0.25 and 0.60, more preferably between 0.30 and 0.50, and most preferably between 0.30 and 0.40.

The natural sweetening composition of the present invention, comprising apple concentrate and Luo Han Guo concentrate wherein the particle size ratio of d(50) of Luo Han Guo concentrate to apple concentrate is between 0.01 to 0.1.

According to this embodiment, particle size ratio of d(50) of Luo Han Guo concentrate to apple concentrate is between 0.01 to 0.1, preferably between 0.02 and 0.08, more preferably between 0.03 and 0.07 and most preferably between 0.03 and 0.06.

According to the special particle sizes and ratios mentioned below, the invention's advantages are reasonable preparing process, high flowability, uniform dispersion of sweetening agent and low moisture absorption. Specifically, this invention of the natural sweetening composition has the advantage of minimal changes in the products' physical characteristics, such as density, flowability, etc.; namely, preventing the final composition from becoming sticky which often occurs when a sweetening agent is added into a composition rather, and retains more of the characteristic flowability and density of the bulked composition with is normally adversely affected by conventional sweetening agent.

In this present invention, the natural sweetening composition further comprising inuline, erythritol, coconut sugar, lactose, locust bean gum; rebaudioside A, rebaudioside B, rebaudioside C, rebaudioside D, rebaudioside E, rebaudioside F, dulcoside A, dulcoside B, rubusoside, isomogroside V, 11-oxomogroside, steviol glycosides, stevia, stevioside, mogroside IV, mogroside V, mogroside VI, siamenoside, monatin and its salts (monatin SS, RR, RS, SR), curculin, glycyrrhizin, glycyrrhizic acid and its salts, thaumatin, monellin, mabinlin, brazzein, hernandulein, phyllodulcin, glycyphyllin, phloridzim, trilobatin, baiyunoside, osladin, polypodoside A, pterocaryoside A, pterocaryoside B, mukurozioside, phlomisoside I, periandrin I, abrusoside A, cyclocarioside I; a sweetener which is extracted from the group comprising Stevia rebaudiana, Glycyrrhiza glabra, Siraitia grosvenorii; sucrose, liquid sucrose, glucose, liquid glucose, fructose, liquid fructose, dextrose, galactose, lactulose, lactose, cellobiose, kojibiose, nigerose, isomaltose, .beta., .beta.-trehalose, .alpha., .beta.-trehalose, tagatose, sophorose, laminaribiose, gentiobiose, turanose, maltose, maltulose, palatinose, gentiobiulose, mannobiose, melibiose, melibiulose, rhamnose, ribose, rutinose, rutinulose, trehalose, xylobiose, xylose, corn syrups, fructo-oligosaccharides; erythritol, glycol, glycerol, threitol, arabitol, xylitol, ribitol, mannitol, sorbitol, dulcitol, iditol, isomalt, maltitol, lactitol, polyglycitol; apple skin extract (applephenon) or mixtures thereof.

In one embodiment, the natural sweetening composition comprising apple concentrate and Luo Han Guo concentrate, inuline.

In another embodiment, the natural sweetening composition comprising apple concentrate and Luo Han Guo concentrate, erythritol.

In another embodiment, the natural sweetening composition comprising apple concentrate and Luo Han Guo concentrate, coconut sugar.

In another embodiment, the natural sweetening composition comprising apple concentrate and Luo Han Guo concentrate, lactose.

In another embodiment, the natural sweetening composition comprising apple concentrate and Luo Han Guo concentrate, locust bean gum.

In one embodiment, the natural sweetening composition comprising apple concentrate and Luo Han Guo concentrate, inuline, erythritol.

In one embodiment, the natural sweetening composition comprising apple concentrate and Luo Han Guo concentrate, inuline, erythritol, coconut sugar.

In one embodiment, the natural sweetening composition comprising apple concentrate and Luo Han Guo concentrate, inuline, erythritol, locust bean gum.

In one embodiment, the natural sweetening composition comprising apple concentrate and Luo Han Guo concentrate, inuline, erythritol, coconut sugar, lactose.

In one embodiment, the natural sweetening composition comprising apple concentrate and Luo Han Guo concentrate, inuline, erythritol, coconut sugar, lactose, locust bean gum.

Use of the natural sweetening composition comprising apple concentrate and Luo Han Guo concentrate in beverage, food, pharmaceutical, oral, dietetic, veterinary or tobacco products

In this present invention, said products are solid, liquid or semisolid.

Use of the natural sweetening composition, wherein;
said beverages are selected from the group comprising colas, ginger ales, root beers, ciders, fruit-flavored soft drinks, powdered soft drinks or mixtures thereof; fruit juices originating in fruits or vegetables, fruit juices including squeezed juices or mixtures thereof, fruit juices containing fruit particles, fruit beverages, fruit juice beverages, beverages containing fruit juices, beverages with fruit flavorings, vegetable juices, juices containing vegetables, and mixed juices containing fruits and vegetables; drinks, sport drinks, energy drinks, near water or mixtures thereof; tea type or favorite type beverages which are coffee, cocoa, chocolate, black tea, green tea, oolong tea or mixtures thereof; beverages containing milk components which are milk beverages, coffee containing milk components, cafe au lait, milk tea, fruit milk beverages, drinkable yogurt, lactic acid bacteria beverages or mixtures thereof; dairy products; alcoholic beverages which are wine, beer, cider; distilled beverages which are spirit, liquor;
said food products are selected from the group comprising bakery products; desserts which are yogurt, jellies, drinkable jellies, puddings, Bavarian cream, blancmange, cakes, brownies, mousse or mixtures thereof; sweetened food products eaten at tea time or following meals; frozen foods; cold confections, types of ice cream which are ice cream, ice milk, lacto-ice or mixtures thereof; ice confections which are sherbets, dessert ices or mixtures thereof; ice cream; general confections, baked confections or steamed confections which are cakes, crackers, biscuits, buns with bean-jam filling or mixtures thereof; rice cakes and snacks; nut and peanut butter; table top products; general sugar confections which are chewing gum, hard candy, soft candy, mints, nougat candy, jelly beans or mixtures thereof; chocolates, chocolate creams; sauces including fruit flavored sauces, chocolate sauces or mixtures thereof; edible gels; cremes including butter cremes, flour pastes, whipped cream or mixtures thereof; jams including strawberry jam, marmalade or mixtures thereof; breads including sweet breads or other starch products or mixtures thereof; spice; general condiments including seasoned soy sauce used on roasted meats, roast fowl, barbecued meat or mixtures thereof, as well as tomato catsup, sauces, noodle broth or mixtures thereof; processed agricultural products, livestock products or seafood; processed meat products which are sausage or mixtures thereof; retort food products, pickles, preserves boiled in soy sauce, delicacies, side dishes; snacks which are potato chips, cookies or mixtures thereof; wafer, waffle, cornets, bars, wafer sheet; cereal products, granola; baby food;
said pharmaceutical products are selected from the group comprising drugs or quasi-drugs that are administered orally or used in the oral cavity, wherein the drug may be in solid, liquid, gel or gas form which is a pill, tablet, spray, capsule, syrup, drop, troche agent, sachet, powder or mixtures thereof; herbal products, vitamins; nutraceutical products comprising any food or part of a food that may provide medicinal or health benefits, including the prevention and treatment of disease;
said oral products are selected from the group comprising hygienic or cosmetic products; personal care products which are oral compositions used in the oral cavity, mouth freshening agents, gargling agents, mouth rinsing agents, toothpaste, tooth polish, dentrifices, mouth sprays, teeth-whitening agents or mixtures thereof;
said dietetic products are selected from the group comprising dietary supplement;
said veterinary products are selected from the group comprising animal feed;
said tobacco products are selected from the group comprising smoke and smokeless tobacco products such as snuff, cigarette, pipe and cigar tobacco, and all forms of tobacco such as shredded filler, leaf, stem, stalk, homogenized leaf cured, reconstituted binders and reconstituted tobacco from tobacco dust, fines or ether sources in sheet, pellet or other forms, tobacco substitutes formulated from non-tobacco materials, dip or chewing tobacco.

In result, the art of sweetener products requires a novelty to provide the advantages of being healthy and natural, not increasing the glycemic index, production simplicity, cost-efficient and sweeter taste because of the synergy between apple concentrate and Luo Han Guo concentrate. It has been found that contrary to prior art and the general knowledge of the skilled person in the natural sweetening composition of comprising apple concentrate having a median particle size (d50) greater than 250 μm and Luo Han Guo concentrate having a median particle size (d50) greater than 5 μm does not show the expected disadvantages in flow behaviour and homogeneity and thus processability if it is provided as particles having a desired particle size distribution. In this case the sweetening composition can be easily handled, mixed with excipients even having a larger particle size and can be processed into beverage, food, pharmaceutical, oral, dietetic, veterinary or tobacco products without the known and expected problems such as segregation or de-mix during the process steps.

According to this invention, some examples are disclosed below:

### Example 1: Chocolate Formulation

| Ingredients | Amount (% by weight) |
|---|---|
| Erythrol | 5-25 |
| Inulin | 20-65 |
| Natural sweetening | |
| composition of apple and | 1-35 |
| LHG concentrates | |
| Hazelnut or puree thereof | 10-30 |
| Cacao products | 1-45 |
| Milk products | 5-30 |
| Vegetable oil | 25-40 |
| Lactose | 0-5 |
| Vanilla | 0-2 |

Production process of the formulation as following:

The mixture of erythrol, natural sweetening composition of apple and LHG concentrates, and inulin is finely mixed and homogenized by means of a mixer. Then, a previously-grinded hazelnut puree is added to this mixture. The resulting mixture is grinded and mixed back so as to be homogenized.

To the preferably pre-ground mixture is added a liquid mixture composed of cacao powder, dry milk products, lactose and vanilla if required, and vegetable oil. Then, conching and settling steps are applied to this mixture, where after the resultant mixture is prepared for sales, following a filling or shaping process as required by the form of the targeted product. The process steps may be illustrated in the following diagram.

In result, thanks to the embodiment disclosed above, dietary chocolate composition comprising natural sweetening composition of apple and LHG concentrates is obtained, with reduced calorie and low glycemic index.

### Example 2: Sweetener Tablet Formulation

| Ingredients | Amount (% by weight) | Function |
|---|---|---|
| Natural sweetening | 5-95 | Sweetener |
| composition of apple and | | |
| LHG concentrates | | |
| D-Mannitol | 50-80 | Filler |
| Croscarmellose sodium | 1-5 | Disintegrant |
| Colloidal silicone dioxide | 0.1-5 | Glidant |
| L-Leucine | 1-10 | Lubricant |
| Sodium stearyl fumarate | 1-5 | Lubricant |

Production process of the formulation as following:

Mannitol, L-leucine and a suitable disintegrant are added to natural sweetening composition of apple and LHG concentrates and mixed. A mixture of colloidal silicone dioxide and sodium stearyl fumarate is added to this first mixture and mixed them together. The resulting mixture is compressed in the form of tablets. The production preferably involves direct compressing method. It can alternatively be produced by wet granulation.

In this formulation, the sweetener is anatural sweetening composition of apple and LHG concentrates, the mannitol used as a filler, and other excipients do not carry any glycemic index potential. Thus, a product is obtained which can be used by those who are in diets or by the diabetics.

Sodium stearyl fumarate is a very efficient lubricant, being less hydrophobic and having a lower delaying effect, when the tablet is dissolved. The present invention involves the use of sodium stearyl fumarate and L-leucine (the lubricant) and colloidal silicone dioxide (the glidant) in the form of a combination, thereby eliminating the problems related to decreasing flowability and to the sticking of these materials to the production apparatuses and elements. Additionally, the product's dissolution rate is brought to a very satisfactory extent, thanks to using croscarmellose sodium as a disintegrant.

### Example 3: Oral Pharmaceutical Formulation

| | Amount | |
|---|---|---|
| Ingredients | (% by weight) | Function |
| Active ingredient | 5-95 | Active ingredient |
| Anhydrous citric acid | 0.1-2 | Flavor enhancer |
| Hypromellose | 0.25-5 | Suspending |
| | | and/or thickening |
| | | agent |
| Natural sweetening composition of | 5-80 | Sweetener |
| apple and LHG concentrates | | |
| Orange aroma | 0.5-2 | Aromatic agent |
| Sodium benzoate | 0.01-0.03 | Microbial |
| | | preservative |
| Disodium EDTA | 0.005-0.1 | Antioxidant |

Production process of the formulation as following:

Active ingredient, hypromellose and anhydrous citric acid are mixed. Natural sweetening composition of apple and LHG concentrates, orange aroma, sodium benzoate and disodium EDTA are added to the first mixture and mixed and homogenized. The resulting mixture is filled into sachets.

1. A natural sweetening composition comprising:
a) Apple concentrate having a median particle size (d50) greater than 100 μm, and
b) Luo Han Guo concentrate having a median particle size (d50) greater than 1 μm.

2. The natural sweetening composition of claim 1, comprising:
a) Apple concentrate having a median particle size (d50) is between 100 and 500 μm, and
b) Luo Han Guo concentrate having a median particle size (d50) is between 1 and 50 μm.

3. The natural sweetening composition of claim 1, comprising:
a) Apple concentrate having a particle size ratio of (d50) to (d90) which is between 0.30 and 0.80, and
b) Luo Han Guo concentrate having a particle size ratio of (d50) to (d90) which is between 0.20 and 0.70.

4. The natural sweetening composition of claim 1, comprising apple concentrate and Luo Han Guo concentrate wherein the particle size ratio of d(50) of Luo Han Guo concentrate to apple concentrate is between 0.01 to 0.1.

5. The natural sweetening composition of claim 1, further comprising inuline, erythritol, coconut sugar, lactose, locust bean gum; rebaudioside A, rebaudioside B, rebaudioside C, rebaudioside D, rebaudioside E, rebaudioside F, dulcoside A, dulcoside B, rubusoside, isomogroside V, 11-oxomogroside, steviol glycosides, stevia, stevioside, mogroside IV, mogroside V, mogroside VI, siamenoside, monatin and its salts (monatin SS, RR, RS, SR), curculin, glycyrrhizin, glycyrrhizic acid and its salts, thaumatin, monellin, mabinlin, brazzein, hernandulein, phyllodulcin, glycyphyllin, phloridzim, trilobatin, baiyunoside, osladin, polypodoside A, pterocaryoside A, pterocaryoside B, mukurozioside, phlomisoside I, periandrin I, abrusoside A, cyclocarioside I; a sweetener which is extracted from the group consisting of Stevia rebaudiana, Glycyrrhiza glabra, Siraitia grosvenorii; sucrose, liquid sucrose, glucose, liquid glucose, fructose, liquid fructose, dextrose, galactose, lactulose, lactose, cellobiose, kojibiose, nigerose, isomaltose, .beta., .beta.-trehalose, .alpha., .beta.-trehalose, tagatose, sophorose, laminaribiose, gentiobiose, turanose, maltose, maltulose, palatinose, gentiobiulose, mannobiose, melibiose, melibiulose, rhamnose, ribose, rutinose, rutinulose, trehalose, xylobiose, xylose, corn syrups, fructo-oligosaccharides; erythritol, glycol, glycerol, threitol, arabitol, xylitol, ribitol, mannitol, sorbitol, dulcitol, iditol, isomalt, maltitol, lactitol, polyglycitol; apple skin extract (applephenon) or mixtures thereof.

6. The natural sweetening composition of claim 5, wherein the composition comprises apple concentrate, Luo Han Guo concentrate and inuline.

7. The natural sweetening composition of claim 5, wherein the composition comprises apple concentrate, Luo Han Guo concentrate and erythritol.

8. The natural sweetening composition of claim 5, wherein the composition comprises apple concentrate, Luo Han Guo concentrate and coconut sugar.

9. The natural sweetening composition of claim 5, wherein the composition comprises apple concentrate, Luo Han Guo concentrate and lactose.

10. The natural sweetening composition of claim 5, wherein the composition comprises apple concentrate, Luo Han Guo concentrate and locust bean gum.

11. The natural sweetening composition of claim 5, wherein the composition comprises apple concentrate, Luo Han Guo concentrate, inuline and erythritol.

12. The natural sweetening composition of claim 5, wherein the composition comprises apple concentrate, Luo Han Guo concentrate, inuline, erythritol and coconut sugar.

13. The natural sweetening composition of claim 5, wherein the composition comprises apple concentrate, Luo Han Guo concentrate, inuline, erythritol and locust bean gum.

14. The natural sweetening composition of claim 5, wherein the composition comprises apple concentrate, Luo Han Guo concentrate, inuline, erythritol, coconut sugar and lactose.

15. The natural sweetening composition of claim 5, wherein the composition comprises apple concentrate, Luo Han Guo concentrate, inuline, erythritol, coconut sugar, lactose and locust bean gum.

16. A beverage, food, pharmaceutical, oral, dietetic or veterinary product comprising a natural sweetening composition comprising apple concentrate and Luo Han Guo.

17. The beverage, food, pharmaceutical, oral, dietetic or veterinary product of claim 16, wherein said product is solid, liquid or semisolid.

18. The beverage, food, pharmaceutical, oral, dietetic or veterinary product of claim 17, wherein:
said beverage is selected from the group consisting of colas, ginger ales, root beers, ciders, fruit-flavored soft drinks, powdered soft drinks or mixtures thereof; fruit juices originating in fruits or vegetables, fruit juices including squeezed juices or mixtures thereof, fruit juices containing fruit particles, fruit beverages, fruit juice beverages, beverages containing fruit juices, beverages with fruit flavorings, vegetable juices, juices containing vegetables, and mixed juices containing fruits and vegetables; drinks, sport drinks, energy drinks, near water or mixtures thereof; tea type or favorite type beverages which are coffee, cocoa, chocolate, black tea, green tea, oolong tea or mixtures thereof; beverages containing milk components which are milk beverages, coffee containing milk components, cafe au lait, milk tea, fruit milk beverages, drinkable yogurt, lactic acid bacteria beverages or mixtures thereof; dairy products; alcoholic beverages which are wine, beer, cider; distilled beverages which are spirit, liquor;
said food product is selected from the group consisting of bakery products; desserts which are yogurt, jellies, drinkable jellies, puddings, Bavarian cream, blancmange, cakes, brownies, mousse or mixtures thereof; sweetened food products eaten at tea time or following meals; frozen foods; cold confections, types of ice cream which are ice cream, ice milk, lacto-ice or mixtures thereof; ice confections which are sherbets, dessert ices or mixtures thereof; ice cream; general confections, baked confections or steamed confections which are cakes, crackers, biscuits, buns with bean-jam filling or mixtures thereof; rice cakes and snacks; nut and peanut butter; table top products; general sugar confections which are chewing gum, hard candy, soft candy, mints, nougat candy, jelly beans or mixtures thereof; chocolates, chocolate creams; sauces including fruit flavored sauces, chocolate sauces or mixtures thereof; edible gels; cremes including butter cremes, flour pastes, whipped cream or mixtures thereof; jams including strawberry jam, marmalade or mixtures thereof; breads including sweet breads or other starch products or mixtures thereof; spice; general condiments including seasoned soy sauce used on roasted meats, roast fowl, barbecued meat or mixtures thereof, as well as tomato catsup, sauces, noodle broth or mixtures thereof; processed agricultural products, livestock products or seafood; processed meat products which are sausage or mixtures thereof; retort food products, pickles, preserves boiled in soy sauce, delicacies, side dishes; snacks which are potato chips, cookies or mixtures thereof; wafer, waffle, cornets, bars, wafer sheet; cereal products, granola; baby food;
said pharmaceutical product is selected from the group consisting of drugs or quasi-drugs that are administered orally or used in the oral cavity, wherein the drug may be in solid, liquid, gel or gas form which is a pill, tablet, spray, capsule, syrup, drop, troche agent, sachet, powder or mixtures thereof; herbal products, vitamins; nutraceutical products comprising any food or part of a food that may provide medicinal or health benefits, including the prevention and treatment of disease;
said oral product is selected from the group consisting of hygienic or cosmetic products; personal care products which are oral compositions used in the oral cavity, mouth freshening agents, gargling agents, mouth rinsing agents, toothpaste, tooth polish, dentrifices, mouth sprays, teeth-whitening agents or mixtures thereof;
said dietetic product is a dietary supplement; or
said veterinary product is an animal feed.

19. A method for making a beverage, food, pharmaceutical, oral, dietetic or veterinary product, the method comprising admixing a natural sweetening composition apple concentrate and Luo Han Guo with the other components of the beverage, food, pharmaceutical, oral, dietetic or veterinary product.

20. The method of claim 19, wherein said product is solid, liquid, or semisolid.

21. The method of claim 19, wherein:
said beverage is selected from the group consisting of colas, ginger ales, root beers, ciders, fruit-flavored soft drinks, powdered soft drinks or mixtures thereof; fruit juices originating in fruits or vegetables, fruit juices including squeezed juices or mixtures thereof, fruit juices containing fruit particles, fruit beverages, fruit juice beverages, beverages containing fruit juices, beverages with fruit flavorings, vegetable juices, juices containing vegetables, and mixed juices containing fruits and vegetables; drinks, sport drinks, energy drinks, near water or mixtures thereof; tea type or favorite type beverages which are coffee, cocoa, chocolate, black tea, green tea, oolong tea or mixtures thereof; beverages containing milk components which are milk beverages, coffee containing milk components, cafe au lait, milk tea, fruit milk beverages, drinkable yogurt, lactic acid bacteria beverages or mixtures thereof; dairy products; alcoholic beverages which are wine, beer, cider; distilled beverages which are spirit, liquor;
said food product is selected from the group consisting of bakery products; desserts which are yogurt, jellies, drinkable jellies, puddings, Bavarian cream, blancmange, cakes, brownies, mousse or mixtures thereof; sweetened food products eaten at tea time or following meals; frozen foods; cold confections, types of ice cream which are ice cream, ice milk, lacto-ice or mixtures thereof; ice confections which are sherbets, dessert ices or mixtures thereof; ice cream; general confections, baked confections or steamed confections which are cakes, crackers, biscuits, buns with bean-jam filling or mixtures thereof; rice cakes and snacks; nut and peanut butter; table top products; general sugar confections which are chewing gum, hard candy, soft candy, mints, nougat candy, jelly beans or mixtures thereof; chocolates, chocolate creams; sauces including fruit flavored sauces, chocolate sauces or mixtures thereof; edible gels; cremes including butter cremes, flour pastes, whipped cream or mixtures thereof; jams including strawberry jam, marmalade or mixtures thereof; breads including sweet breads or other starch products or mixtures thereof; spice; general condiments including seasoned soy sauce used on roasted meats, roast fowl, barbecued meat or mixtures thereof, as well as tomato catsup, sauces, noodle broth or mixtures thereof; processed agricultural products, livestock products or seafood; processed meat products which are sausage or mixtures thereof; retort food products, pickles, preserves boiled in soy sauce, delicacies, side dishes; snacks which are potato chips, cookies or mixtures thereof; wafer, waffle, cornets, bars, wafer sheet; cereal products, granola; baby food;
said pharmaceutical product is selected from the group consisting of drugs or quasi-drugs that are administered orally or used in the oral cavity, wherein the drug may be in solid, liquid, gel or gas form which is a pill, tablet, spray, capsule, syrup, drop, troche agent, sachet, powder or mixtures thereof; herbal products, vitamins; nutraceutical products comprising any food or part of a food that may provide medicinal or health benefits, including the prevention and treatment of disease;
said oral product is selected from the group consisting of hygienic or cosmetic products; personal care products which are oral compositions used in the oral cavity, mouth freshening agents, gargling agents, mouth rinsing agents, toothpaste, tooth polish, dentrifices, mouth sprays, teeth-whitening agents or mixtures thereof;
said dietetic product is a dietary supplement; or
said veterinary product is an animal feed.
